(19) United States
(12) Patent Application Publication
(10) Pub. No.: US 2010/0118270 A1
(43) Pub. Date: May 13, 2010

(54) ADAPTOR FOR MOUNTING A GONIO LENS ONTO A HAND-HELD EYE EXAMINATION DEVICE

(75) Inventors: William Shea, Pleasanton, CA (US); Yeou-Yen Cheng, Saratoga, CA (US); Shelly Reed, Dublin, CA (US); Willem Crone, Oroville, CA (US); Barry Linder, Danville, CA (US); Phillip Baker, Walnut Grove, CA (US); Yan Zhou, Pleasanton, CA (US)

Correspondence Address: LAW OFFICE OF CHARLES E. KRUEGER, P.O. BOX 5607, WALNUT CREEK, CA, 94596-1607, US

(73) Assignee: CLARITY MEDICAL SYSTEMS, INC., Pleasanton, CA (US)

(21) Appl. No.: 12/606,993

(22) Filed: Oct. 27, 2009

Related U.S. Application Data
(60) Provisional application No. 61/111,994, filed on Nov. 6, 2008.

(51) Int. Cl. A61B 3/117

(52) U.S. Cl. 351/219

(57) ABSTRACT

An apparatus and method is disclosed that provides a means for mounting a separate optical element, such as a gonio lens, to a hand-held eye imaging device. In this way, the doctor only needs to use one hand to hold the imaging apparatus while the other hand can be freed to open the patient eye lid and also maintain a safe relative distance of the gonio lens with respect to the patient's eye. The gonio lens and adaptor can each or both be made disposable and also mechanically switchable relative to each other as well as relative to the handheld eye imaging device so that different portions of the patient eye can be imaged sequentially without the need to rotate the hand held eye imaging device.

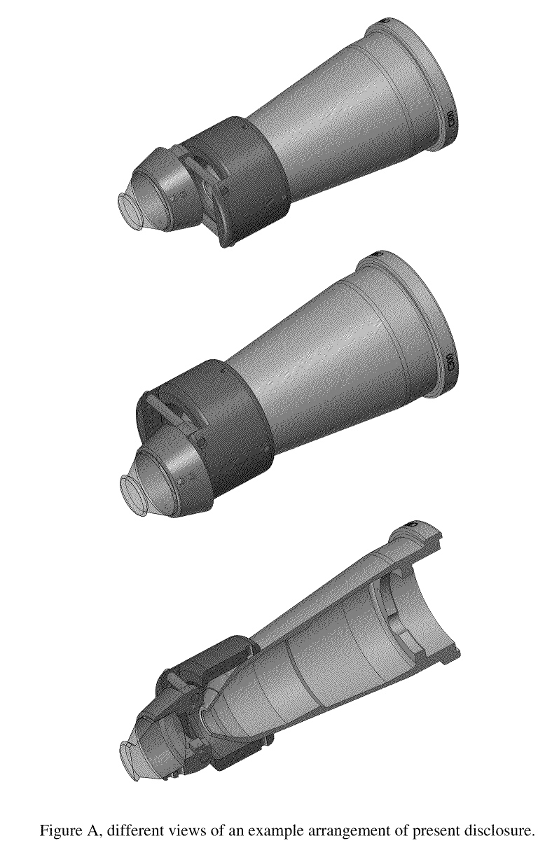

### RELATED APPLICATIONS

This application claims priority from a provisional application entitled ADAPTOR FOR MOUNTING A GONIO LENS ONTO A HAND-HELD EYE EXAMINATION DEVICE, Application No. 61/111,994, filed Nov. 6, 2008, which is hereby incorporated by reference for all purposes.

### TECHNICAL FIELD

The present disclosure relates generally to imaging apparatus for eye imaging/examination.

### BRIEF DESCRIPTION OF THE DRAWINGS

Figure A illustrates the one example of the disclosed means for mounting a detachable gonio lens onto a handheld eye imaging device.

### DESCRIPTION OF EXAMPLE EMBODIMENTS OVERVIEW

This disclosure describes a means for mounting a separate or detachable optical element such as an off the shelf (OTS) optical lens or a gonio lens with mirror facet(s), disposable or non-disposable to a hand-held eye imaging device with the help of a mechanical adaptor. In an example embodiment, when gonioscopy imaging is being conducted, the doctor only needs to use one hand to hold the imaging apparatus, while the other hand can be freed to open the patient eye lid and/or to maintain a safe distance between the gonio lens and the patient's eye. The adaptor can be made disposable and also mechanically switchable so that the detachable optical element (such as a gonio lens) can be moved to different positions relative to the handheld eye imaging device to enable different portions of the eye to be sequentially imaged without the need to rotate the handheld eye imaging device which may have been ergonomically designed for hand holding with a certain orientation.

Reference will now be made in detail to various embodiments of the invention. Examples of these embodiments are illustrated in the accompanying drawings. While the invention will be described in conjunction with these embodiments, it will be understood that it is not intended to limit the invention to any embodiment. On the contrary, it is intended to cover alternatives, modifications, and equivalents as may be included within the spirit and scope of the invention as defined by the appended claims. In the following description, numerous specific details are set forth in order to provide a thorough understanding of the various embodiments. However, the present invention may be practiced without some or all of these specific details. In other instances, well known process operations have not been described in detail in order not to unnecessarily obscure the present invention. Further, each appearance of the phrase an “example embodiment” at various places in the specification does not necessarily refer to the same example embodiment.

Traditional gonioscopy is usually conducted using a slit lamp with the patient placing his/her chin on a chin rest and the doctor holding a gonio lens against the eye of the patient while looking through the slit lamp to directly observe the angle region of the patient's eye. The gonio lens can have one, two three or four mirrored facets to direct illumination and imaging light to the angle region of the anterior chamber of the patient eye. Usually, such a slit lamp based gonioscopy imaging requires the use of the doctor's two hands. The doctor needs to use one hand to hold the handle of the gonio lens and maybe laterally translate the gonio lens relative to the patient eye in order to see a large area of one section of the anterior angle, while his/her other hand needs to be used to open or hold the patient's eye lid or to adjust the optics of the slit lamp.

When a handheld eye imaging system such as the RetCam system is used for anterior chamber angle imaging, there are two possible approaches that can be taken. The RetCam system is manufactured by the assignee of the present application and includes a light weight video camera in a plastic or metal holder that facilitates the connection of changeable lenses. Details of the RetCam system are available Claritymsi website with the extension .com/downloads/RetCam.pdf. The first approach is to use a wide angle RetCam lens piece to directly image the anterior chamber angle region with the handheld assembly substantially tilted with respect to the optical axis of the eye and in doing so, there is a need for an optical coupling gel to fill the relatively large wedge gap between the patient eye and the front lens surface of the handheld assembly. A problem with this approach is that the optical coupling gel tends to flatten if the patient is lying down on his/her back or the gel tend to drip off if the patient is sitting upright. A second approach is to place a gonio lens between the patient eye and the handheld eye imaging device so that the handheld eye imaging device can be held relatively normally with respect to the eye to leave only a small gap to be either filled by the eye tear or a small amount of optical coupling gel which can be thin and easy to wipe off afterwards. However, with this second alternative approach, there is a need to mechanically attach the gonio lens to the handheld assembly as otherwise, the doctor has to use one hand to hold the gonio lens and the other hand to hold the handheld imaging device, which will make it difficult for the doctor to properly control a safe distance between the eye, the gonio lens and the hand held imaging device.

Various example embodiment, including apparatuses and methods are disclosed that enables a handheld eye imaging device such as the RetCam system to view an image (anterior or posterior of the eye) through a separate optical element (contact or non-contact gonio lens, Iridectomy lens, for examples) without the need to hold both the separate optical element and the eye imaging system with two hands. In the case of a gonio lens, for example, depending on the number of mirror facets made on the gonio lens, there may be a need to laterally translate the gonio lens from one side to the other side with respect to the handheld eye imaging device such as a slit lamp or fundus camera so that the anterior chamber angle image can be moved to the central region of the field of view of the image sensor. On the other hand, if there is only one mirrored facet on the gonio lens, there may be a need to rotate the gonio lens off-center into 4 quadrant positions relative to the handheld eye imaging device in order to capture 4 quadrant section images of the anterior chamber angle of the patient eye. Note that the off center translation of the gonio lens relative to the handheld eye imaging device, such as the RetCam imaging system, will not only move the anterior chamber angle image towards the center of the field of view but also produce a larger and better view of the angle region of the patient's eye. The presently invented apparatuses and methods can mechanically hold the separate optical element to be combined with the handheld imaging system at the correct spatial requirements to optimize the anatomical image onto the system's image sensor. This example embodiment could also allow the change in orientation of the separate optical element to that of the patient/system, by either a lateral, rotational or angular movement/displacement. This example embodiment could also provide an optical coupler between the handheld imaging system and separate optical element, while at the same time provide the desired/optimal angular, rotational, or spatial arrangement and orientations. Another aspect of the invention is to make either the separate optical element such as the gonio lens, or the adaptor, or both the separate optical element and the adaptor, disposable. In this way, cross contamination among different patients can be minimized. Still another aspect of the present invention is that the separate optical element and the adaptor can be snapped or attached to the handheld imaging device without the need for other tools.

This example embodiment optimizes the viewing and digital imaging capability of existing products, while at the same time providing a more consistent, controlled, and easier implementation/use of acquiring digital images. In addition, this device can facilitate better digital documentation of an eye's anatomy, whereas most lenses require a two-hand operation with analog imaging devices.

This example embodiment is an improvement to existing methods and products, which combines two separate devices, one that can acquire an image with one that provides direct analog (human) visualization over a greater area of anatomy. The combination of the two, results in the ability to acquire high-quality digital images over a greater area of anatomy.

One method of attachment for the gonio adapter that may be implemented is by making a groove around the periphery of the RetCam lens piece. This would provide the rotational adjustment and position the separate lens and adaptor assembly at the proper distance with the proper orientation relative to the RetCam lens piece. There may be positioning detents machined into this groove so that the gonio lens and the adaptor assembly can lock into two or more positions 90 or 180 degrees apart from each one another. For a one mirror faceted gonio lens, four positions separated 90 degrees apart can be selected. For a two mirror faceted gonio lens, two positions separated by 180 degrees can be selected to enable the gonio lens to achieve a lateral translation relative to the RetCam lens piece. Meanwhile, 4 or more positions separated by any orientation angles can also be selected to enable the handheld eye imaging device to capture multiple images of the eye. The separate optical element can also be mounted onto two rods and thus can slide along those rods, providing the required off center translation of the gonio lens with respect to the RetCam lens piece. In the illustration as shown in Figure A, the machined groove is not shown on the RetCam lens body, it is held in place with setscrews for prototyping ease. However, it is preferred that the attachment of this adapter to the RetCam lens piece is without the use of tools. The adapter may snap into a groove around the RetCam lens housing. The use of a groove will allow the gonio lens to be rotated about the RetCam lens piece with ease.

A second alternate method is to attach the gonio lens and the adaptor assembly via a series of slots that runs along the length of the RetCam lens piece. There can, for example, be 4 of these slots, separated 90 degrees apart, around the body of the RetCam lens piece. The gonio lens mounted on an adaptor as an assembly can slide into these slots and there can be a detent at the end of each slot to lock the adaptor into place. Rotation of the gonio lens would be accomplished by pulling it off the RetCam lens piece, rotating the assembly 90 degrees, and re-inserting it into the slots.

A third example method to rotate the gonio is to mount a single faceted or mirrored gonio lens on a rotating ring where the lens and the ring do not rotate in relation to each other. The preferred viewing axis, and offset, of the gonio is aligned to the optical axis of the RetCam, or other lens. There can be four detents, 90 degrees apart, to position the gonio lens. Generally, optical components are rotationally symmetric; the gonio lens can be constructed so that this is not the case. If the gonio lens outside diameter, or mounting surface, provides the offset, then as the lens is rotated about its mechanical axis it would provide the correct views. Another advantage to this is that at different offset, or angle, the gonio lens can be placed into the same adapter. The lens provides the correct offset, not the mechanical components.

An alternate method of translation and rotation is to use three eccentric ring(s). This would require a groove, either machined into the RetCam lens piece or attached onto it, as the guide for rotation. Into this groove are three rings. The middle ring has its outside diameter and inside diameter off set (eccentric) by ½ the amount of travel required per the desired off center distance for the gonio lens relative to the center of the RetCam lens piece. Rotation of this middle ring will provide the translational travel to the gonio lens. The inner ring, outer ring and the gonio are keyed to rotate together. Rotation of these elements will provide the two halves or 4 quadrant views required for examination. The relatively rotatable rings should have detents so that the gonio lens is aligned at 90 degree steps. An alternate method of locking the gonio lens into place may be the use of magnets as the detents. In addition, ring rotating segment can have a toric inner and outer radius that can permit rotation as well as angular tilt in order to optimize the view relayed by the gonio optics through the RetCam. An angular adjustment of the optical axis will facilitate a fine tuning to improve the view of the individual features of the anterior and most importantly the various anatomical structures of the angle as well as views of various implants or post-surgical results for the clinicians further evaluation.

Any of the above-mentioned mechanical structures may be machined into the RetCam lens piece housing at initial manufacture or they may be retrofitted as an applied ring to the body of the RetCam lens piece housing. This ring may be mounted by adhesive or mechanically.

Another method to provide transversal travel of the gonio lens with respect to the RetCam lens piece is to use parallel arms. There can be one arm mounted on each side of the gonio lens. These arms, at the other end, can be fixed to the body of the RetCam lens piece or the adapter ring. Located at one end, or both ends, would be a detent to lock the rotation and/or translation of these arms at the point that they have traveled the correct distance for the required amount of travel for the gonio lens. This detent snap into place, letting the operator know that it has traveled the correct distance.

A variation of the parallel arm travel is to use arms that are not parallel. The arms would converge at a point in front of the gonio lens. This would rock the gonio lens as it travels to its two positions, required an asymmetric lens. The advantage to this is that the front portion of the adapter can be much smaller than the other designs, with the exception of the eccentric ring design.

One preferred embodiment of the present disclosure is that part of the gonio lens or the gonio lens or the gonio lens and the adapter assembly be made disposable. There are several ways to make this implementable. The gonio lens can be a injection molded plastic element and in such a case, it can be removed from the adaptor and discarded after each use. The gonio may have a snap-into-place feature that attaches itself to the adapter. One feature that may be useful for the gonio lens is the use of a soft front contact cup. The portion of the gonio lens that actually contacts the eye may be made of some resilient optical material to improve patient comfort and safety in the case of sudden eye movement. The contact cup may be a hydrogel, silicone or similar molded component. The contact cup may be the portion that is disposable in this system. It may have other mechanical features, gel or releasable adhesive that would lock it into place. The front portion of the gonio lens can also be a thin layer of optically transparent coupling film or a gel/fluid filled bag or simply a gel that can be later wiped off the eye. This optically transparent layer can be attached to the rest of the gonio lens through various means such as liquid filled vacuum suction and this would reduce the cost of the replacement component. Still another possibility is to make the gonio lens and the adaptor assembly disposable by, for example, making this whole assembly with plastic injection molding.

In order to improve the accuracy and repeatability of the images captured from the same eye, additional features can be added to overlap the image taken. For example, when the anterior segment image is recorded, the position of the pupil and some features on the retina, possibly the fovea, macula, or the optical nerve head can also be simultaneously recorded. This information could be overlaid during the following eye examinations to align the handheld imaging system. While the overlaid image might be software selected so that the pupil edge and some high contrast landmarks on the retina surface are viewed, an alternative can be to have a reticle that may have concentric rings to be made inside the gonio lens or the adaptor or the RetCam lens piece as long as the reticle is roughly in a conjugate plane to the image sensor plane. In this way, the reticle can be used to align the pupil and/or a landmark on the retina, such as the macula, fovea or optical nerve head, which may be different for the left eye and right eye. The gonio lens or the adaptor or the assembly of the two can also be optically designed to have spatially different imaging properties so that while a certain portion of the field of view will produce a well focused anterior chamber angle image, another portion of the field of view will at the same time produce a well focused fundus or retina image. This aspect of the invention can be used to align the combined eye imaging apparatus to be square to the eye and/or to be centered and rotationally positioned properly before an image is taken. It would also allow the registration of the images taken at different times to be relatively repeatable. This repeatability can be more important for a handheld eye imaging device than a bench top device due to lack of patient support and positioning.

The presently disclosed apparatus and method can be applied to any optical imaging system and a separate optical element that can be attached to the system to enhance the performance of the system. For examples, the system could be adapted to a microscope's ocular “port”; integrated into a slit-lamp tower; adapted to gonio lenses; the separate optical element could be integrated into the system's lens; and the design features of the separate optical element could also be integrated in the system;

In addition to optometrist and ophthalmologist practices; the presently described example embodiments can effectively be applied to anywhere as long as the visualization of an eye's anatomy is desired.

The invention has now been described with reference to the example embodiments. Alternatives and substitutions will now be apparent to persons of skill in the art. For example, the example embodiments have been described with reference to the RetCam system but other handheld video systems can also be utilized. Accordingly, it is not intended to limit the invention except as provided by the appended claims.

What is claimed is:

1. A means for attaching a separate optical element onto a hand held eye imaging device comprising:
a separate optical element,
an adaptor, and
a handheld eye imaging device

2. The means of claim 1 wherein the separate optical element is a gonio lens.

3. The means of claim 1 wherein the hand held eye imaging device is EyeCam or RetCam eye imaging system.
(other claim points)
The adaptor can be snapped onto the body of the handheld eye image device without the need for a tool.
The gonio lens and adaptor assembly can be rotated with respect to the handheld eye imaging device
The gonio lens and adaptor assembly can be snapped into different positions with respect to the handheld eye imaging device.
The gonio lens can be transversally moved off center with the desired orientation and lock into positions relative to the adaptor.
The gonio lens can be rotated off center with the desired orientation and lateral displacement, and mechanically locked into positions relative to the adaptor.
The gonio lens can be made disposable and snapped onto the adaptor.
The gonio lens or the gonio lens and the adaptor assembly can be made disposable.
The front portion of the gonio lens that makes contact with the eye can be made disposable.
The gonio lens or the adaptor or the handheld eye imaging device can have reticle made within to help align the eye relative to these land marks.
The gonio lens can be designed to have spatially different imaging characteristics to simultaneously provide clears images of both the anterior and the posterior of the eye.
